(19) United States
(12) Patent Application Publication
(10) Pub. No.: US 2023/0255739 A1
(43) Pub. Date: Aug. 17, 2023

(54) MULTIMODALITY FOLDABLE MULTIPLE SMALL ANIMAL HOLDER

(71) Applicant: Bruker Belgium N.V., Kontich (BE)

(72) Inventor: Timothy Claessens, Scherpenheuvel (BE)

(73) Assignee: Bruker Belgium N.V., Kontich (BE)

(21) Appl. No.: 18/151,446

(22) Filed: Jan. 8, 2023

(30) Foreign Application Priority Data
Feb. 17, 2022 (EP) 22 157 224.1

(51) Int. Cl. A61D 3/00; A61D 7/04

(52) U.S. Cl. CPC A61D 3/00; A61D 7/04

(57) ABSTRACT

A holding device for simultaneously holding several small animals includes at least three beds each for a small animal. Each bed is attached to a respective holder element. The holding device is geometrically constructed and mechanically configured where two of the holder elements can be moved together with their respective beds above a third holder element without changing the vertical orientation of the beds being attached to the two holder elements. This provides a compact holding device for a multitude of small animals facilitating flexible and easy access to each small animal when attached to the holder for generating a 3D image of all animals at the same time, e.g., in an MRI, PET, CT scanner or the like. The holding system is easy to use, in particular for rats or mice, and can easily be applied in a wide variety of locations through its small and compact construction.

### CROSS-REFERENCE TO RELATED APPLICATIONS

This application claims priority to European Patent Application

EP 22 157 224.1 filed Feb. 17, 2022, the entire contents of which are hereby incorporated in full by this reference.

### DESCRIPTION

### Field of the Invention

The invention relates to a holding device for simultaneously holding several small animals comprising at least three beds each for a small animal, whereby each bed is attached to an according holder element.

### BACKGROUND OF THE INVENTION

Holding devices for simultaneous holding a multitude of beds with small animals thereon are for example described in U.S. Pat. No. 8,660,633 B2 (Reference [1]) or in Esparza-Cross 2008: “Wireless self-gated multiple-mouse cardiac cine MRI”, Mag. Resonance in Medicine 59, 1203-1206 (2008) (Reference [2]).

### TECHNICAL BACKGROUND OF THE INVENTION

In general, the present invention relates to the technical field of laboratory support for conducting scientific investigations on—in most cases living—animals. In particular, the invention contributes as an auxiliary aid for holding small animals, like e.g., mice and rats, during examinations.

U.S. Pat. No. 10,632,271 B1 (Reference [3]) describes a “versatile subject bed” for use with one or more test subjects that is configured to keep the subjects sedated throughout either a surgical procedure or imaging process. This subject bed can include interchangeable manifolds allowing the platform to hold up to four subjects at the same time. The four-subject manifold can include an elevated platform that holds two subject side by side and allows the subjects to be stacked during imaging or other procedures.

In Reference [2] cited above, four mice can be inserted geometrically in parallel into an MRI imaging device. However, these mouse holders are mechanically separated from each other.

Reference [1] also cited above describes a generic holding device for simultaneously holding several small animals. Here, each small animal can be separately fixed to a bed and inserted into a common holder which provides anesthesia ports. The animal beds can all be attached to the holder but must be inserted separately into the holder. The animal beds can be separated from the holder for individually placing the small animals. After mounting, the beds each have a fixed position relative to the holder and to each other. In particular, the device according to Reference [1] does not enable any possibility of moving the beds with respect to each other.

Although there is a wide range of already existing solutions according to prior art devices for holding a multitude of small animals at the same time, no one of them offers the combination of a compact device and an easy animal access when mounted to the holder.

### SUMMARY OF THE INVENTION

### Objects of the Invention

It is an object of the present invention to provide—without much technical effort—a generic and compact holding device for a multitude of small animals facilitating a flexible, easy access to each small animal when attached to the holder, in particular for generating a 3D image of all animals at the same time, e.g., in an MRI, PET, CT scanner or the like. The holding system should be easy to use, in particular for rats or mice, and easily applied in a wide variety of locations through its small and compact construction.

### BRIEF DESCRIPTION OF THE INVENTION

This object is achieved, in accordance with the present invention and in a surprisingly simple and effective way, by modifying a generic device for simultaneously holding several small animals as defined above in the first paragraph in that the holding device is geometrically constructed and mechanically configured such that two holder elements can be moved together with their respective beds above a third holder element without thereby changing the vertical orientation of the beds being attached to the two holder elements.

The holding device according to the invention is adapted for use as a multiple-purpose animal bed to be able to measure/scan multiple small animals in multiple modalities:

It can receive e.g., 4 mice or maybe only 2 mice and a rat or larger mouse.

It can be used in stretched/unfolded mode with e.g., 4 mice near each other, being arranged horizontally or rather in folded mode with e.g., 4 mice arranged in a circle.

In stretched/unfolded mode, the beds are aligned in a common plane and the bottom surfaces as well.

Both modes can be mechanically locked.

While unfolding or folding the bed holder, the orientation of the beds is always in the same vertical direction; therefore, no mouse will accidentally fall off.

The unfolding mechanism provides enough space for anesthesia supply.

Although the holding device according to the present invention is preferably used in devices for preclinical imaging, there are also other possible and useful implementations of the invention in further fields of research.

Preferred embodiments and further developments of the invention:

In straightforward embodiments of the invention, the holding device is geometrically constructed and configured such that the two holder elements can be moved above the third holder element by a translatory movement.

The holding device is designed to be stable due to the translatory adjustability of the holding elements and is a compact arrangement of the animals' beds. Small animals are kept in horizontal position once the beds/holders are arranged for cross-sectional scanning i.e., tomographic modalities. Only that allows for arranging the small animals like that. For surface scanning (optical, thermography) they need to be arranged next to each other.

However, in some other cases of application, there might be some need to avoid translatory movement. For these cases, alternative embodiments of the invention are suitable, where the two holder elements can be moved above the third holder element by a rotating movement.

A rotating movement allows a compact setup of the holding device and reduces the footprint in the unfolded position. This might apply if there is no need to keep the beds attached to the holder horizontal, e.g., when used in a scanner for material samples, e.g., bore cores.

In particularly preferred variants of these embodiments of the invention, the two holder elements which can be moved above the third holder element are pivotably linked to the third holder element each with at least two holder arms.

By using two holder arms, the mechanical stability during rotation and in the static case is improved.

In another class of advantageous embodiments of the present invention, the holding device has a locking mechanism which is geometrically constructed and mechanically configured such that two of the holder elements can be locked in their movement and fixed in a folded position with at least one of the first two holder elements being placed above the third holder element and in an unfolded position with at least one of the first two holder elements being placed horizontally beside the third holder element.

This mechanism makes the holder mechanically stable in both, the open and closed state, and a small animal can be easily removed without removing a bed from the holder. It prevents the assembly of moving unintendedly during a cross-sectional scanning—maybe induced by an animal moving. Further, this makes sure that for animal preparation—in unfolded position—the assembly does not fold and can be put on a flat surface without toppling over.

In a preferred further development of this embodiment, the holder arms of the two holder elements have deepenings and the third holder element has spring loaded balls which can engage in the deepenings so that the two holder elements can be fixed with respect to the third holder element in the folded position or in the unfolded position.

As a result, the fixation of the two retaining elements in the unfolded and folded positions can be achieved without much force and with a smooth transition. It is the least space consuming way of providing a locking mechanism. Only a single spring-loaded ball is needed to lock a holder element into folded and unfolded position.

A preferred variant of this further development is characterized in that the deepenings have a slot-like structure.

This allows for relaxed requirements on manufacturing tolerances of the holding device, as the deepenings of the holder arms do not need to be aligned exactly with the spring-loaded balls when being fixed in the folded or unfolded position. Compared to a circular deepening a slot-like deepening allows more freedom of the ball of the spring-loaded ball to snap into. In that sense, it allows to compensate for manufacturing tolerances.

Alternatively or conjointly, in a further advantageous variant, the third holder element comprises two spring loaded balls, which engage in the deepening of the upper holder arms and the lower holder arms in the unfolded and in the folded position, respectively.

This reduces material usage as only two spring loaded balls are required for the device to lock into folded and unfolded position, which means less material costs and less mechanical complexity.

A further embodiment of the invention is characterized in that the first and second holder element each have a flat surface at their bottom such that the first and second holder element can stand stable on a flat working surface, in particular on a desk, in an unfolded position with at least one of the first and second holder element being placed horizontally beside the third holder element.

Due to this measure, the device is stable when open and small animals can be removed or inserted freehand. As explained above, this arrangement will make sure that for animal preparation—in unfolded position—the assembly does not fold and can be put on a flat surface without toppling over.

Also favorable are embodiments of the invention, which are characterized in that in a completely folded position, with the first and the second holder element being both placed above the third holder element, the holding device forms a cylindrical shape with circular circumference.

Due to the circular cylinder shape, the device is designed to be particularly compact. A cylindrical shape is especially helpful for cross-sectional imaging.

Preferred is a further development of these embodiments, in which the holder device is constructed in such a way that its cylindrical circumference in the completely folded state fits into an entry opening of an imaging instrument with circular inner diameter.

This would be e.g., a diameter of around 80 mm for an in-vivo micro-CT device or 85-200 mm for a magnetic resonance imaging instrument. It is an important requirement that the device needs to fit into the bore for imaging.

In some embodiments, the third holder element comprises two beds for small animals, in particular mice.

This allows to maximize the number of animals to be arranged occupying the least amount of space. It is required for an application which demands a total of 4 beds to be arranged for simultaneous scanning.

In alternative embodiments, the third holder element comprises one bed for a small animal, in particular a rat.

This makes the holding device more flexible to accommodate one smaller and one larger animal and is required to provide enough space even for a larger animal.

In another class of comparatively compact and simple embodiments of the invention, each of the animal beds have a semicircular cross section.

That feature provides a stable holding of each small animal. Further, a semicircular cross section provides enough space for the small animal. In principle, other cross-sections are also possible, but a semicircular cross section is easy to handle and clean since there are no corners compared to rectangular cross sections. In addition, a semicircular cross-section will provide enough space to put a warming pad below the small animal.

Further beneficial embodiments of the invention are characterized in that the beds at their free ends adjacent to their respective holder elements have a holding bar or stopper element preventing the small animal from slipping out of its bed.

The small animals are thereby kept in position even if the holding device is tilted when transferring it, e.g., to an imaging instrument. Further, this prevents fluids (e.g., mouse urine) from dropping out of the bed and polluting the imaging system.

Preferred is a further development of these embodiments, in which the holding bars or stopper elements have a notch for holding a tail of the small animal while lying in the bed.

In this way, the small animals are at least partially fixed in an upward or downward position. In addition, this might act as a defined outlet for fluids.

Still other advantageous embodiments of the holding device according to the present invention are characterized in that each of the holder elements have a slot or slit for receiving and holding inlet tubes for leading anesthetization gas to a gas mask for the respective small animal.

This arrangement allows for a compact design of the entire assembly, since no inlet tubes need to be routed outside the perimeter of the holding device. It can be combined with an afore mentioned anesthesia mask.

Further advantages can be extracted from the description and the enclosed drawings. The features mentioned above and below can be used in accordance with the invention either individually or collectively in any combination. The embodiments mentioned are not to be understood as exhaustive enumeration but rather have exemplary character for the description of the invention.

### BRIEF DESCRIPTION OF THE DRAWINGS

The invention is shown in the drawings and is explained in more detail based on illustrative embodiments.

In the drawings:

FIG. 1a shows a stereoscopic view of an embodiment of the holding device according to the invention in a folded position of the holder elements and four beds attached thereto with a mouse conveniently lying on one of the upper beds having its tail hanging over a notch of a stopper element;

FIG. 1b shows a schematic horizontal front view on the holding device as shown in FIG. 1a in direction looking from the beds to the holder elements;

FIG. 2a shows in a stereoscopic view the embodiment of FIG. 1a in an unfolded position with the attached beds arranged side by side in a horizontal plane;

FIG. 2b shows a schematic horizontal front view on the holding device in its unfolded position as shown in FIG. 2a looking in direction from the beds to the holder elements;

FIG. 3a shows a schematic vertical cross section through a holding device in the unfolded position as shown in FIG. 2a, displaying holder arms pivotably linking the holder elements;

FIG. 3b shows a schematic vertical cross section through a holding device as shown in FIG. 3a, however in a partially folded position;

FIG. 3c shows a schematic horizontal front view on a holding device as shown in FIG. 3a in its completely folded position looking in direction from the beds to the holder elements, the holding device having only three beds, namely two smaller beds in the upper position and a bigger bed in the lower position;

FIG. 4a shows a schematic stereoscopic view of an embodiment of the holding device according to the invention having four beds and being in a partially folded position;

FIG. 4b shows an enhanced part of the embodiment of FIG. 4a displaying screws with spring loaded balls engaged in according deepenings;

FIG. 4c shows a schematic stereoscopic view of an embodiment of the invention similar to the holding device as shown in FIG. 4a in a partially folded position; and

FIG. 4d shows a schematic, semi-transparent horizontal front view on the holding device as shown in FIG. 4c in a partially folded position displaying holder arms pivotably linking the holder elements.

### Detailed Description of the Preferred Embodiments

The present invention is principally concerned with providing tools for performing scientific investigations on small animals, in particular with the development of an improved holding device for a multitude of small animals like mice and rats.

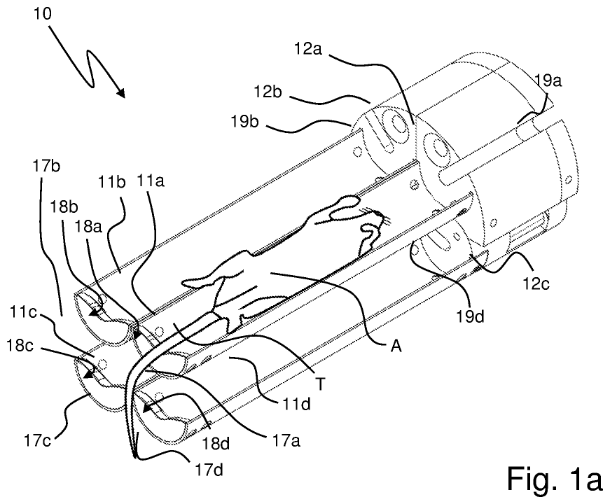

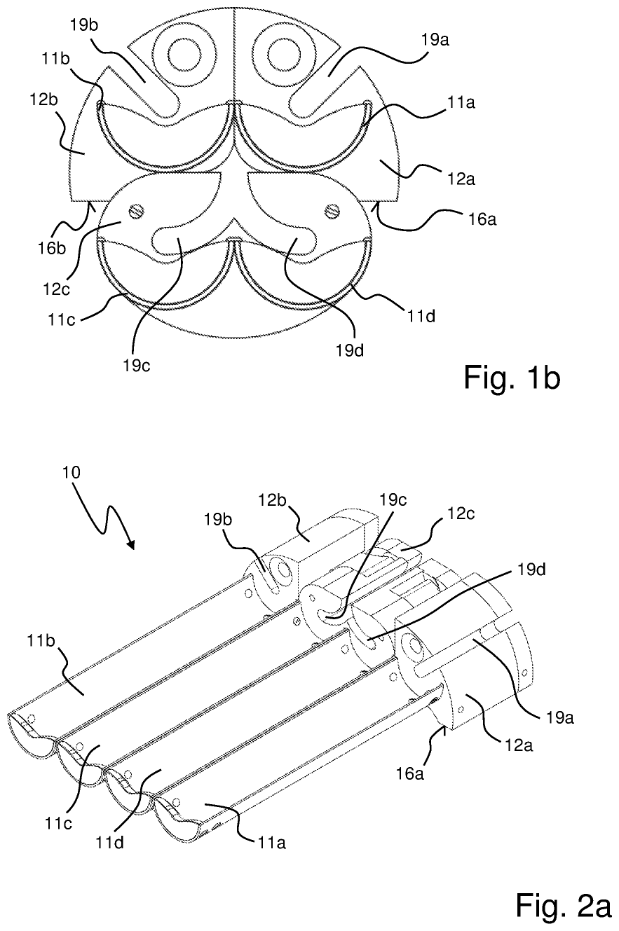

FIG. 1a schematically depicts a holding device 10 for simultaneously holding several small animals A comprising at least three beds 11a, 11b, 11c, 11d each for a small animal A, whereby each bed 11a, 11b, 11c, 11d is attached to an according holder element 12a, 12b, 12c.

According to the invention, this holding device 10 is geometrically constructed and mechanically configured such that two of the holder elements 12a, 12b can be moved together with their respective beds 11a, 11b above a third holder element 12c without thereby changing the vertical orientation of the beds 11a, 11b being attached to the two holder elements 12a, 12b.

The holding device 10 of FIG. 1a is depicted in a folded position of the holder elements 12a, 12b, 12c with two beds 11a, 11b attached to the two holder elements 12a, 12b in the upper position and another two beds 11c, 11d attached to the third holder element 12c in the lower position. The two holder elements 12a, 12b can be moved above the third holder element 12c by a rotating movement in this embodiment.

The beds 11a, 11b, 11c, 11d in this embodiment each have a semicircular cross section. In a completely folded position with the first and the second holder element 12a, 12b being both placed above the third holder element 12c the holding device 10 forms a cylindrical shape with circular circumference. This provides the biggest amount of animals in a circle and in this way one can easily use circular scan methods like CT, PET etc.

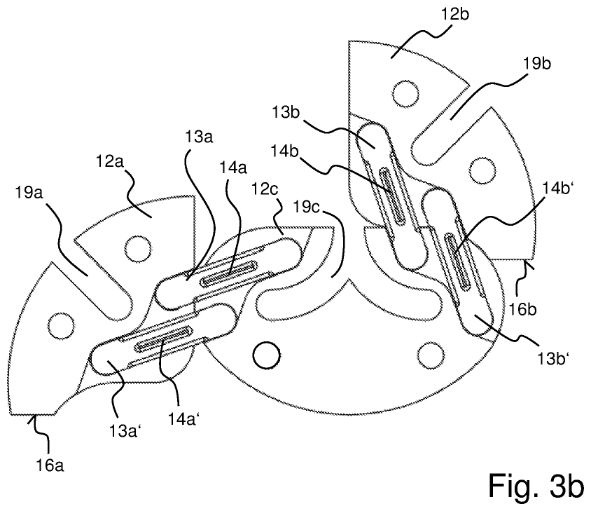

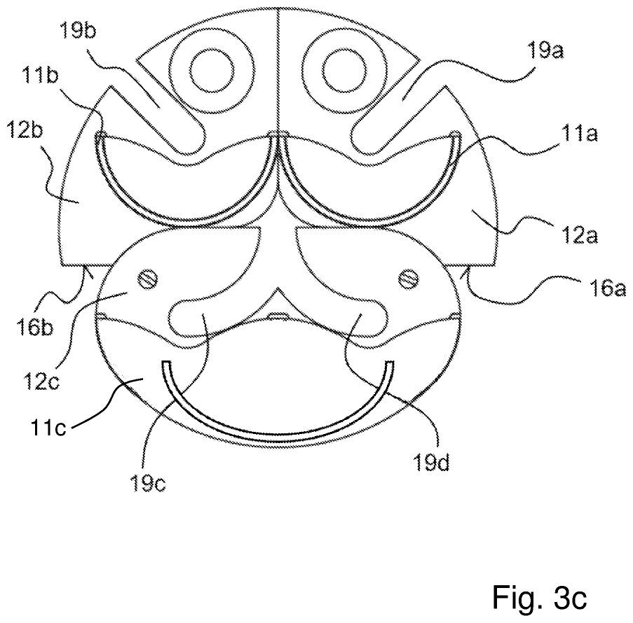

The third holder element 12c in this embodiment comprises two beds 11c, 11d for small animals A, in particular mice. In other embodiments of the invention (as shown in FIG. 3c), the third holder element 12c comprises only one bed 11c for a small animal A, in particular a bigger mouse or a rat.

Further, holding bars or stopper elements 17a, 17b, 17c, 17d are provided, each having a notch 18a, 18b, 18c, 18d for holding a tail T of the small animal A while lying on its bed 11a, 11b, 11c, 11d. The holder elements 12a, 12b, 12c each have a slot or slit 19a, 19b, 19c, 19d for receiving and holding inlet tubes for leading anesthetization gas to a gas mask for the respective small animal A.

FIG. 1b shows a schematic horizontal front view on the holding device as shown in FIG. 1a in direction looking from the beds 11a, 11b, 11c, 11d to the holder elements 12a, 12b, 12c.

The first and second holder element 12a, 12b each have a flat surface 16a, 16b at their bottom such that the first and second holder element 12a, 12b can stand stable on a flat working surface, in particular on a desk, in an unfolded position with at least one of the first and second holder element 12a, 12b being placed horizontally beside the third holder element 12c.

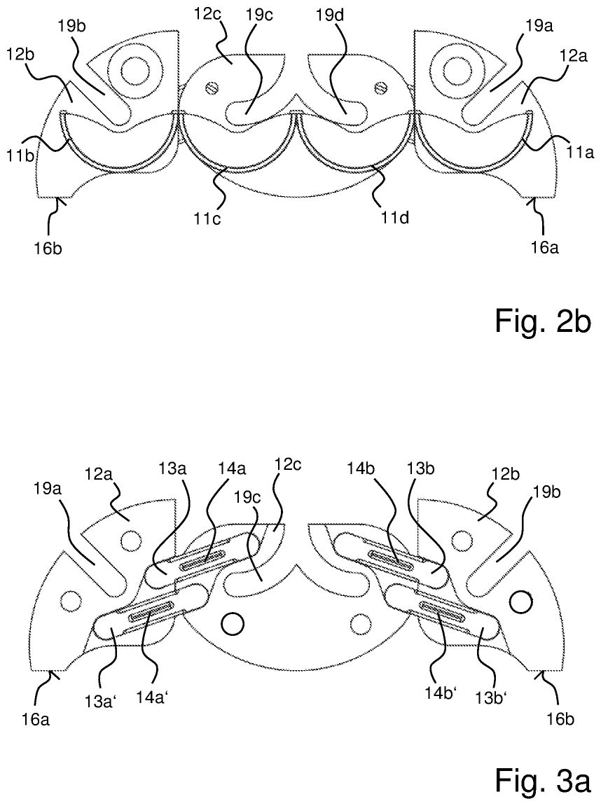

FIG. 2a shows in a stereoscopic view the holding device 10 of FIG. 1a in the unfolded position with the attached beds 11a, 11b, 11c, 11d arranged side by side in a horizontal plane. In this unfolded, stretched position, up to four small animals can be easily arranged in the holding device 10 or removed therefrom after measurement. In this flat setup, one can easily prepare four mice or scan in an optical scanner, if 2D images are needed and one cannot scan through multiple mice at the same time.

FIG. 2b shows in a schematic horizontal front view the holding device 10 in its unfolded position as depicted in FIG. 2a, looking again in a direction from the beds 11a, 11b, 11c, 11d to the holder elements 12a, 12b, 12c. Flat surfaces 16a, 16b are aligned with lower portion of the third holder element 12c to ensure a stable positioning of the holder on a flat surface.

FIG. 3a displays a schematic vertical cross section through a holding device 10 in its unfolded position as shown in FIG. 2b. The two holder elements 12a, 12b can again be rotationally moved above the third holder element 12c and are pivotably linked to the third holder element 12c each with at least two holder arms 13a,13a′; 13b, 13b′. These holder arms make sure that the folding movement is made in such a way that the orientation of the beds is always vertical.

The holding device 10 has a locking mechanism which is geometrically constructed and mechanically configured such that two of the holder elements 12a, 12b in their movement can be locked and fixed in a folded position with at least one of the first and second holder element 12a, 12b being placed above the third holder element 12c and in an unfolded position with at least one of the first and second holder element 12a, 12b being placed horizontally beside the third holder element 12c.

The holder arms 13a, 13a′; 13b, 13b′ of the two holder elements 12a, 12b have deepenings 14a, 14a′; 14b, 14b′ with a slot-like structure.

FIG. 3b shows a schematic vertical cross section through a holding device 10 as shown in FIG. 3a, however in a partially folded position of the holder element 12b on the right side. Holder arms 13a, 13a′; 13b, 13b′ are attached to the respective holder elements 12a, 12b and 12c such that they remain parallel during and after the rotary movement.

FIG. 3c shows a schematic horizontal front view on a holding device 10 as shown in FIG. 3a in its completely folded position looking in direction from the beds 11a, 11b, 11c to the holder elements 12a, 12b, 12c. In this embodiment, the holding device 10 has only three beds 11a, 11b, 11c, namely two smaller beds 11a, 11b in the upper position and a bigger bed 11c in the lower position. Now, the small animals are arranged over 120 degrees instead off 90 degrees like it is with the 4-mice formation as e.g., shown in FIGS. 1a and 1b.

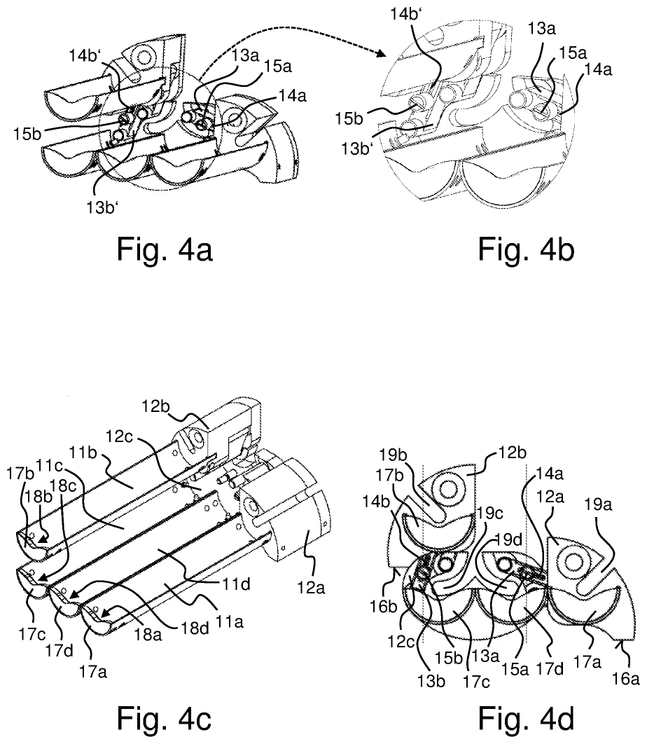

A schematic stereoscopic view of an embodiment of the holding device according to the invention having four beds and being in a partially folded position with one of the beds in the lifted position and the other three beds in the lower horizontal arrangement is depicted in FIG. 4a.

The third holder element 12c carrying two beds 11c, 11d comprises screws with spring loaded balls 15a, 15b which can engage in the deepenings 14a, 14b′ of the holder arms 13a, 13b′ so that the first two holder elements 12a, 12b can be fixed with respect to the third holder element 12c in the folded position or in the unfolded (stretched) position. In FIGS. 4a-4d, a portion of holder element 12c has been omitted to better reveal the location and function of the screws with spring loaded balls 15a, 15b and the deepenings 14a, 14b′ of the respective holder arms.

FIG. 4b shows an enhanced part of the embodiment of FIG. 4a displaying the screws with spring loaded balls 15a, 15b engaged in according deepenings 14a, 14b′.

More precisely, FIG. 4b merely depicts an adjustable frame for the spring-loaded balls, i.e., the screws 15a, 15b, the former not being visible in this picture. In this embodiment, the spring pressure can be adjusted by means of a screwdriver (not displayed here) or the like. FIGS. 2b, 3c and show an opening for accessing these screws from the front of the holder. This allows easy adjustment of the holding pressure of the spring-loaded balls.

The third (bottom) holder element 12c contains two ball screws, which are inserted from the backside. Once the holder arm 13b′ moves over the spring-loaded ball screw, the ball snaps into the grove 14b′ and will lock the movement of the arm 13b′. That means, the spring-loaded ball screws 15a, 15b are not part of the holder arms, but part of and rigidly connected to the third (bottom) holder element 12c. In fact, there is a little hole in the third (bottom) holder element 12c on the front (bed) side that allows to adjust the fit of the ball screw and thus of the load onto the holder arm. In the figure, the third (bottom) holder element is hidden and reveals the inserted ball screws and it looks like they are connected to the arms—which they are not in fact. But the figures nicely illustrate that the ball snaps into the grove of the top holder arm in case the assembly is open and into to bottom holder arms once it is closed.

The stereoscopic view of FIG. 4c shows an embodiment of the invention similar to the holding device as shown in FIG. 4a in the same partially folded position.

Finally, FIG. 4d shows a schematic, semi-transparent front view on the holding device as shown in FIG. 4c in its partially folded position displaying holder arms 13a, 13b pivotably linking the first holder elements 12a, 12b to the third holder element 12c.

### LIST OF REFERENCE SIGNS

10 holding device
11a, 11b, 11c, 11d beds
12a, 12b first and second holder elements
12c third holder element
13a; 13b upper holder arms
13a′; 13b′ lower holder arms
14a,14a′; 14b, 14b′ deepenings
15a, 15b screws with spring loaded balls
16a, 16b flat surfaces
17a, 17b, 17c, 17d holding bars or stopper elements
18a, 18b, 18c, 18d notches
19a, 19b, 19c, 19d slots or slits
A small animal
T tail

### PRIOR ART CITATIONS

Publications considered for assessing patentability of the present invention:
[1] U.S. Pat. No. 8,660,633 B2
[2] Esparza-Cross 2008: “Wireless self-gated multiple-mouse cardiac cine MRI”, Mag. Resonance in Medicine 59, 1203-1206 (2008)
[3] U.S. Pat. No. 10,632,271 B1

What is claimed is:

1. A holding device configured for simultaneously holding several small animals, comprising:
at least three beds each for a small animal;
wherein each bed of the at least three beds is attached to a respective holder element;
wherein the holding device is geometrically constructed and mechanically configured wherein two of the holder elements can be moved together with their respective beds above a third holder element without changing a vertical orientation of the beds being attached to the two holder elements.

2. The holding device according to claim 1, wherein the two holder elements can be moved above the third holder element by a translatory movement.

3. The holding device according to claim 1, wherein the two holder elements can be moved above the third holder element by a rotating movement.

4. The holding device according to claim 3, wherein the two holder elements which can be moved above the third holder element are pivotably linked to the third holder element each with at least two holder arms.

5. The holding device according to claim 1, wherein the holding device has a locking mechanism which is geometrically constructed and mechanically configured wherein two of the holder elements in their movement can be locked and fixed in a folded position with at least one of the first and second holder element being placed above the third holder element and in an unfolded position with at least one of the first and second holder element being placed horizontally beside the third holder element.

6. The holding device according to claim 5, wherein the holder arms of the two holder elements have deepenings and the third holder element has spring loaded balls which can engage in the deepenings wherein the two holder elements can be fixed with respect to the third holder element in the folded position or in the unfolded position.

7. The holding device according to claim 6, wherein the deepenings have a slot-like structure.

8. The holding device according to claim 6, wherein the third holder element comprises two spring loaded balls which engage in the deepening of the upper holder arms and the lower holder arms in the unfolded and in the folded position, respectively.

9. The holding device according to claim 1, wherein the first and second holder element each have a flat surface at their bottom wherein the first and second holder element can stand stable on a flat working surface in an unfolded position with at least one of the first and second holder element being placed horizontally beside the third holder element.

10. The holding device according to claim 1, wherein in a completely folded position with the first and the second holder element being both placed above the third holder element the holding device forms a cylindrical shape with circular circumference.

11. The holding device according to claim 1, wherein the third holder element comprises two beds for small animals, in particular mice.

12. The holding device according to claim 1, wherein the third holder element comprises one bed for a small animal, in particular a rat.

13. The holding device according to claim 1, wherein the beds each have a semicircular cross section.

14. The holding device according to claim 1, wherein holding bars or stopper elements are provided, each having a notch for holding a tail of the small animal while lying on its bed.

15. The holding device according to claim 1, wherein the holder elements each have a slot or slit for receiving and holding inlet tubes for leading anesthetization gas to a gas mask for the respective small animal.
